(19) United States
(12) Patent Application Publication
(10) Pub. No.: US 2011/0301118 A1
(43) Pub. Date: Dec. 8, 2011

(54) Methods of treatment utilising glucan formulations

(75) Inventor: Reinhard Koenig, Rockville, MD (US)

(73) Assignee: NOVOGEN RESEARCH PTY LTD, North Ryde, NSW (AU)

(21) Appl. No.: 13/124,157

(22) PCT Filed: Oct. 15, 2009
(86) PCT No.: PCT/AU09/01361

§ 371 Date: Aug. 30, 2011

Related U.S. Application Data
(60) Provisional application No. 61/105,525, filed on Oct. 15, 2008.

(51) Int. Cl. A61K 31/716; A61P 17/00; A61B 19/02; A61P 17/02

(52) U.S. Cl. 514/54; 206/438

(57) ABSTRACT

The present invention relates to methods for the treatment of skin wounds or lesions, and connective tissue damage or injury, comprising administering to a subject an effective amount of a glucan composition. Also provided are methods for the promotion of wound healing and tissue regeneration.

### FIELD OF THE INVENTION

The present invention relates generally to the administration of glucan and compositions comprising the same for promoting wound healing and for the treatment of skin disorders and connective tissue disease or injury.

### BACKGROUND OF THE INVENTION

Glucans are oligosaccharides or polysaccharides composed predominantly or wholly of glucose. Glucans are widely distributed in nature, being found in the cell walls of a variety of plants, fungi and microorganisms. Beta-(1,3)(1,6) glucans derived from yeast, such as the bakers yeast Saccharomyces cerevisiae, have been identified as having particular therapeutic potential for the treatment of a variety of disorders and conditions. Beta-glucans act to enhance the immune system, stimulating the activity of the primary defence cells, natural killer cells, neutrophils and macrophages. As such beta-glucans play a role in combating infection. Various beta-glucans have also been implicated in, for example, the treatment of cancer, septic shock, arthritis and in wound healing and reducing cholesterol.

A microparticulate beta-(1,3)(1,6) glucan from Saccharomyces cerevisiae, the isolation of which is described in U.S. Pat. No. 6,242,594, has been found to be therapeutically effective when administered, for example, to subjects suffering from a bone fracture, ulcers caused by physical trauma, surgical wounds, impaired blood flow, infections or neoplasia, or in persons in need of enhancement of fixation of implanted orthopaedic devices to bone.

Accordingly, there is considerable interest in the development of suitable efficacious formulations comprising glucans to maximise benefit to patients.

The present invention is predicated on the inventor's determination of advantageous dosage regimens of glucan containing compositions.

### SUMMARY OF THE INVENTION

According to a first aspect of the present invention there is provided a method for the treatment of a skin wound or lesion, or connective tissue damage or injury, the method comprising administering to a subject an effective amount of a glucan composition comprising from about 0.01% (10 μg/ml) to about 10% (10 mg/ml) glucan.

The glucan composition may comprise from about 0.02% (20 μg/ml) to about 10% (10 mg/ml) glucan. The glucan composition may comprise from about 0.02% (20 μg/ml) to about 5% (5 mg/ml) glucan. The glucan composition may comprise from about 0.05% (50 μg/ml) to about 2% (2 mg/ml) glucan. In one embodiment the glucan composition comprises about 0.1% (100 μg/ml) glucan. In another embodiment the glucan composition comprises about 1% (1 mg/ml) glucan.

Typically the glucan is derived from yeast cell walls. The glucan may be a particulate or microparticulate glucan. In an embodiment the glucan is a microparticulate branched beta-(1,3)(1,6) glucan. In an embodiment the glucan is microparticulate poly-(1,3)-beta-D-glucopyranosyl-(1,6)-beta-D-glucopyranose.

The glucan may be administered topically to the skin of the subject.

In an embodiment the glucan is administered in the form of a cream or gel to the skin of the subject. In another embodiment the glucan is administered in the form of a dressing or bandage into which the glucan has been incorporated.

In an embodiment administration of the glucan composition is at least once daily. In an embodiment the administration is at least once daily for at least three days, at least four days, or at least five days.

The skin wound or lesion may be a surgical wound or result from physical damage, injury or trauma. The skin wound or lesion may be, for example, a burn, ulcer, incision, puncture, abrasion or laceration. The skin wound or lesion may be the result of laser or chemical peeling. The skin wound or lesion may result from skin graft removal, for example split-skin graft removal.

The skin wound or lesion may be acute or chronic. In an embodiment the skin wound or lesion is an acute wound or lesion.

The treatment may be therapeutic and/or cosmetic.

The subject may require reconstructive or cosmetic surgery, and the glucan composition may be administered before, during or after the surgery. The surgery may comprise a skin graft procedure.

According to a second aspect there is provided a method for the treatment of a skin wound or lesion, or connective tissue damage or injury, the method comprising administering to a subject an effective amount of a glucan composition comprising from about 0.01% (10 μg/ml) to about 10% (10 mg/ml) glucan, wherein the glucan composition is administered at least once daily for at least five days.

In one embodiment the glucan composition comprises about 0.1% (100 μg/ml) glucan. In another embodiment the glucan composition comprises about 1% (1 mg/ml) glucan.

According to a third aspect there is provided a method for promoting tissue regeneration in a subject, the method comprising administering to a subject an effective amount of a glucan composition comprising from about 0.01% (10 μg/ml) to about 10% (1 mg/ml) glucan.

In an embodiment the tissue is skin. In an embodiment the subject is undergoing or has undergone a skin graft procedure and the glucan composition promotes tissue regeneration thereby facilitating or promoting incorporation of the skin graft into the surrounding tissue of the subject.

According to a fourth aspect there is provided a method for promoting wound healing in a subject, the method comprising administering to a subject an effective amount of a glucan composition comprising from about 0.01% (10 μg/ml) to about 10% (10 mg/ml) glucan.

According to a fifth aspect there is provided a method for promoting the growth of dermal tissue in a tissue culture comprising the same, comprising exposing the tissue culture to an effective amount of a glucan composition comprising from about 0.01% (10 μg/ml) to about 10% (10 mg/ml) glucan.

In an embodiment the dermal tissue is grown to produce tissue for grafting onto a burn, surgical wound, acute wound or chronic wound. The dermal tissue may be derived from a subject in need of a tissue graft.

According to a sixth aspect there is provided use of an effective amount of a glucan composition comprising from about 0.01% (10 μg/ml) to about 10% (10 mg/ml) glucan for the therapeutic and/or cosmetic treatment of a skin wound or lesion, or connective tissue damage or injury.

According to a seventh aspect there is provided use of the daily application of an effective amount of a glucan composition containing from about 0.01% (10 μG/ml) to about 10% (10 mg/ml) glucan for the treatment of a skin wound or lesion, or connective tissue damage or injury, or the promotion of healing thereof.

According to an eighth aspect there is provided a unit dose treatment product for the treatment of a skin wound or lesion, or connective tissue damage or injury, or the promotion of healing thereof, the product comprising multiple individual containers each containing a unit dose of a glucan composition, the glucan composition comprising from about 0.01% (10 mg/ml) to about 10% (10 mg/ml) glucan, and wherein the product is designed for the sequential application of the components of the individual containers, preferably once a day for at least 5 consecutive days.

### BRIEF DESCRIPTION OF THE DRAWINGS

The present invention will now be described, by way of non-limiting example only, with reference to the accompanying drawings in which:

FIG. 1 shows the test sites for administration of glucan composition on the right (A) and left (B) side of a Goettingen mini-pig prior to dermal ablation and the test sites on the right (C) and left (D) side immediately after dermal ablation.

FIG. 2 shows histologic differences in unlasered, untreated control skin (A) of a Goettingen mini-pig and laser ablated skin of a Goettingen mini-pig after treatment with Vaseline® (B) and 0.1% Glyc-101™ (C). The treated skin was subjected to a daily application of either Vaseline® or 0.1% Glyc-101™ for 5 days post laser ablation. The skin biopsies were stained with hematoxylin and eosin and are shown at ×400 magnification.

FIG. 3 shows histologic differences in the laser ablated skin of a Goettingen mini-pig after treatment with: 0.1% Glyc-101™ on days 1,3 and 5 post laser ablation (A); 0.1% Glyc-101™ daily for 5 days post laser ablation (B); and placebo daily for 5 days post laser ablation (C). The skin biopsies were stained with hematoxylin and eosin and are shown at ×400 magnification.

FIG. 4 shows the test sites on the side of a Goettingen mini-pig for administration of placebo, 0.1% Glyc-101™ and 1.0% Glyc-101™ at (A) Day 1 after the split-skin graft; (B) Day 1 post dose of a uniform application to a maximum thickness of 1 mm daily; C and D) Day 2 pre dose; (E and F) Day 3 pre dose; (G and H) Day 4 pre dose; (I and J) Day 5 pre final dose; (K and L) Day 7; and (M and N) Day 10.

FIG. 5 shows histologic differences in split-skin graft skin of a Goettingen mini-pig after Day 1 of treatment with (A) placebo; (B) 0.1% Glyc-101′; and (C) 1.0% Glyc-101™ The skin biopsies were stained with hematoxylin and eosin and are shown at ×400 magnification.

FIG. 6 shows histologic differences in split-skin graft skin of a Goettingen mini-pig after Day 2 of daily treatment with (A) placebo; (B) 0.1% Glyc-101™; and (C) 1.0% Glyc-101™. The skin biopsies were stained with hematoxylin and eosin and are shown at ×400 magnification.

FIG. 7 shows histologic differences in split-skin graft skin of a Goettingen mini-pig after Day 3 of daily treatment with (A) placebo; (B) 0.1% Glyc-101™; and (C) 1.0% Glyc-101™. The skin biopsies were stained with hematoxylin and eosin and are shown at ×400 magnification.

FIG. 8 shows histologic differences in split-skin graft skin of a Goettingen mini-pig after Day 5 of daily treatment with (A) placebo; (B) 0.1% Glyc-101™; and (C) 1.0% Glyc-101™. The skin biopsies were stained with hematoxylin and eosin and are shown at ×400 magnification.

FIG. 9 shows histologic differences in split-skin graft skin of a Goettingen mini-pig after Day 10 after daily treatment for 5 days with (A) placebo; (B) 0.1% Glyc-101™; and (C) 1.0% Glyc-101™. The skin biopsies were stained with hematoxylin and eosin and are shown at ×400 magnification.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein, the term “effective amount” is understood to mean an amount or dose of a molecule or composition sufficient to achieve the desired therapeutic or cosmetic result whilst at least partially avoiding unwanted side effects. Thus in the context of the present disclosure the term “effective amount” may also be referred to as, and should be considered to encompass, an “effective dose”. The effective amount or dose of a molecule or composition will vary with, for example, the age and other physical characteristics and condition of the subject to be treated, the severity of the condition being treated/prevented, the extent of any microbial infection, the identity of any microorganism(s) present, the duration of the treatment, the mode of administration, the specific molecule or compositions employed and the concentration of active molecule(s) in the composition administered. Thus, it is not possible to specify an exact “effective amount” or effective dose. However, for any given case, an appropriate “effective amount” or dose may be determined by one of ordinary skill in the art using only routine experimentation.

As used herein the term “glucan” includes a glucan molecule(s) in a purified, partially purified or substantially purified form, a glucan present as a cellular extract, and a glucan present in a composition or formulation. Thus, for the present purposes, the glucan may be associated with one or more additional components, which components may or may not constitute active agents in their own right.

As used herein the term “subject” includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. Even more typically, the mammal is a human.

As used herein the terms “treating”, “treatment”, “preventing” and “prevention” refer to any and all uses which provide a therapeutic and/or cosmetic benefit to a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As used herein, the term “therapeutic” or “therapeutic effect” refers to a molecule or composition which when provided to a subject, provides a beneficial physiological effect. The beneficial effect may be, for example, the reduction, elimination, or prevention of a condition or disease, one or more symptoms of the condition or disease, or one or more side effects of the condition or disease. The beneficial effect need not solely be therapeutic but may also be at least partially cosmetic in nature.

As used herein, the term “cosmetic” or “cosmetic effect” refers to a composition which when provided to a subject serves a primarily aesthetic purpose in enhancing or improving physical appearance. The beneficial effect need not solely be cosmetic but may also be at least partially therapeutic in nature.

As used herein in the context of wound healing and tissue regeneration, the terms “promoting”, “promotion” and variations thereof refer to the ability of a molecule or composition to induce, enhance or otherwise advance the natural processes associated with wound healing and tissue regeneration. In embodiments the promotion may be relative to the healing or regeneration observed in the absence of administration of the molecule or composition. The promotion may be direct or indirect. It will be understood that in indirectly promoting wound healing or tissue regeneration, the molecule or composition may effect the expression or activity of molecules which themselves regulate or otherwise influence, either directly or indirectly, the wound healing or tissue regeneration processes. The promotion may be qualitative, quantitative and/or temporal. That is, for example, the administration of the molecule or composition may result in more rapid wound healing or tissue regeneration than would occur in the absence of such administration. Furthermore, by “promotion” it will be understood that the administration of the molecule or composition may result in healing or regeneration such that the skin wound or lesion, or connective tissue damage or injury, heals with less scarring and/or fibrosis, less collagen deposition and more superficial surface area than in the absence of such administration.

As exemplified herein, it has now been demonstrated that beneficial therapeutic and cosmetic outcomes can be achieved with daily application of glucan compositions comprising concentrations of glucan between about 0.1% (100 μg/ml) and 1% (1 mg/ml).

Accordingly, one aspect of the invention provides a method for the treatment of a skin wound or lesion, or connective tissue damage or injury, the method comprising administering to a subject an effective amount of a glucan composition comprising from about 0.01% (10 μg/ml) to about 10% (10 mg/ml) glucan. Also provided is a method for the treatment of a skin wound or lesion, or connective tissue damage or injury, the method comprising administering to a subject a glucan composition comprising from about 0.01% (10 μg/ml) to about 10% (10 mg/ml) glucan, wherein the glucan composition is administered at least once daily for at least five days.

Further provided are methods for promoting tissue regeneration and promoting wound healing, the methods comprising administering to subjects in need an effective amount of a glucan composition comprising from about 0.01% (10 μg/ml) to about 10% (10 mg/ml) glucan. The invention also provides a unit dose treatment product for the treatment of a skin wound or lesion, or connective tissue damage or injury, or the promotion of healing thereof, the product comprising multiple individual containers each containing a unit dose of a glucan composition, the glucan composition comprising from about 0.01% (10 μg/ml) to about 10% (10 mg/ml) glucan, and wherein the product is designed for the sequential application of the components of the individual containers, preferably once a day for at least 5 consecutive days.

Typically the glucan is a microparticulate glucan, more typically a microparticulate branched beta-(1,3)(1,6) glucan such as poly-(1,3)-beta-D-glucopyranosyl-(1,6)-beta-D-glucopyranose. The glucan may be a microparticulate glucan prepared in accordance with the process as described and claimed in U.S. Pat. No. 6,242,594 (Kelly; the disclosure of which is incorporated herein by reference in its entirety). U.S. Pat. No. 6,242,594 describes the isolation of a microparticulate poly-(1,3)-beta-D-glucopyranosyl-(1,6)-beta-D-glucopyranose from Saccharomyces cerevisiae, which glucan has the general structure

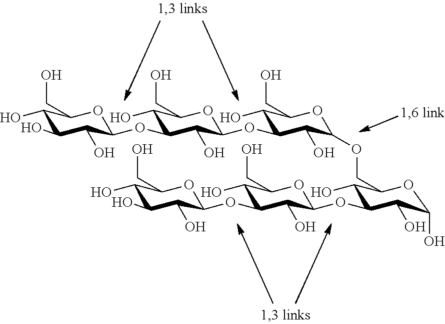

This, as an active pharmaceutical ingredient (API) is termed herein Glucoprime™ or Glyc-101™. These terms may be used interchangeably herein. This substance typically has a molecular weight of between about 1.2 million and 2.2 million Daltons and is an amorphous powder slightly soluble in most aqueous and organic solvents but sparingly soluble in DMSO.

Whilst exemplified herein, those skilled in the art will appreciate that the scope of the present invention is not limited to the glucan described in U.S. Pat. No. 6,242,594 or a glucan produced in accordance with the methods described therein.

The skilled addressee will also appreciate that the specific dosing regimen (with respect for example to frequency and duration of administration) to be employed in accordance with embodiments of the invention may be determined on a case-by-case basis, for example, by the treating physician. Such determinations are well within the capabilities of those skilled in the art without undue burden or experimentation. Exemplified herein in respect of acute skin wounds, treatment once a day for at least three days, at least four days or at least five days has been shown to be desirable. However the dosing regimen may be modified for particular patients taking into consideration, for example, the nature of the wound, lesion or injury suffered (for example depending on whether the wound, lesion or injury is acute or chronic), the severity thereof, the glucan composition administered, and the desired outcome.

Further, it will be understood that the specific dose level of a composition of the invention for any particular individual will depend upon a variety of factors including, for example, the activity of the glucan employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. In accordance with particular embodiments of the invention the glucan composition comprises from about 0.01% (10 μg/ml) to about 10% (10 mg/ml) glucan, or more typically from about 0.02% (20 μg/ml) to about 5% (5 mg/ml) or from about 0.05% (50 μg/ml) to about 2% (2 mg/ml). Alternatively, the composition may comprise about 0.03% (30 μg/ml), 0.04% (40 μg/ml), 0.05% (50 μg/ml), 0.06% (60 μg/ml), 0.07% (70 μg/ml), 0.08% (80 μg/ml), 0.09% (90 μg/ml), 0.1% (100 μg/ml), 0.2% (200 μg/ml), 0.3% (300 μg/ml), 0.4% (400 μg/ml), 0.5% (500 μg/ml), 0.6% (600 μg/ml), 0.7% (700 μg/ml), 0.8% (800 μg/ml), 0.9% (900 μg/ml), 1.0% (1 mg/ml), 1.1% (1.1 mg/ml), 1.2% (1.2 mg/ml), 1.3% (1.3 mg/ml), 1.4% (1.4 mg/ml) or 1.5% (1.5 mg/ml) glucan.

In accordance with the aspects and embodiments of the present invention the subject may be suffering from a skin wound or lesion, or a connective tissue disease or injury, the treatment of which may be effected by the administration of a glucan, typically a microparticulate beta-(1,3)(1,6) glucan. The wound may be a surgical wound or a wound resulting from physical damage, injury or trauma including, for example a burn wound, pressure sore, bed sore, burn, puncture, incision, abrasion, laceration or other wound requiring closure; ultraviolet light-induced skin damage; infection; skin wrinkle or blemish; tissue defect following trauma or surgery; connective tissue damage or injury including injuries to tendons and ligaments. Thus, embodiments disclosed herein apply to the treatment of chronic and acute wounds and the scope of the present disclosure is not intended to be limited by reference to any particular wound type.

Skin grafts are often used to promote the healing of a variety of wounds including, for example, surgical or burn wounds, varicose ulcers (venous ulcers), pressure ulcers (bedsores), diabetic ulcers or to reconstruct skin removed during surgery. Skin graft procedures are typically performed during reconstructive or cosmetic surgery. Embodiments of the invention disclosed herein find application in promoting the repair of wound sites generated by the removal of skin for a skin graft procedure and in the promotion of integration of a skin graft into the surrounding tissue of a subject undergoing or having undergone a skin graft procedure. Skin graft procedures to which embodiments disclosed herein relate include, for example, autografts, allografts, xenografts, artificial skin grafts, composite grafts, full thickness skin grafts and split-skin grafts. Those skilled in the art will appreciate that embodiments of the invention find application in the treatment of wounds and wound sites and the promoting of wound healing and tissue regeneration before, during or after any form of reconstructive or cosmetic surgery.

Glucan preparations as disclosed herein may also be used in the ex vivo promotion of tissue growth. For example, it may be desirable to isolate dermal tissue (e.g. skin) from a subject requiring a dermal tissue graft (or a donor individual in the case of an allograft or xenograft) and to culture the isolated tissue in the presence of a glucan preparation to promote tissue growth prior to use of the cultured tissue in a graft procedure.

Glucan may be administered in accordance with the present invention in the form of pharmaceutical compositions, which compositions may comprise one or more pharmaceutically acceptable carriers, excipients or diluents. Such compositions may be administered in any convenient or suitable route such as by topical, parenteral, or oral routes. Typically for the purposes of achieving the therapeutic and cosmetic benefits as disclosed herein for use on skin wounds and lesions, the route of administration may be topical. Alternatively, administration by injection, for example subcutaneous injection, may also be appropriate depending on the desired outcome. However those skilled in the art will appreciate that the appropriate mode of administration will, at least in part, depend upon the nature of the condition to be treated. In circumstances where it is required that appropriate concentrations of the desired agent are delivered directly to the site in the body to be treated, administration may be regional rather than systemic. Regional administration provides the capability of delivering very high local concentrations of the desired agent to the required site and thus is suitable for achieving the desired therapeutic or preventative effect whilst avoiding exposure of other organs of the body to the compound and thereby potentially reducing side effects.

The compositions of the invention typically comprise one or more pharmaceutically acceptable carriers, excipients or diluents. Glucans may further be combined with other therapeutic or cosmetic agents for example, but not limited to, antibiotics, antimicrobial agents, antiseptics, anaesthetics, moisturisers or cosmetic bases.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Topical formulations typically comprise an active ingredient together with one or more acceptable carriers, and optionally any other therapeutic ingredients. Formulations suitable for topical administration include liquid or semi-liquid preparations suitable for penetration through the skin to the site of where treatment is required, such as gels, creams, ointments, pastes, lotions or liniments.

By way of example only, the composition for topical administration may comprise, as exemplified herein, glucan in microparticulate form, in a composition comprising ethanol, triethanolamine, Carbopol® 980 NF, titanium dioxide and purified water. The resulting composition may be a highly viscous, aqueous gel suitable for topical administration. Alternatively for topical administration, the composition may be, for example, in the form of a cream, ointment, paste, lotion, liniment, aerosol or other sprayable composition, which composition will typically be suitable for direct application to the site of the wound, lesion, damage or injury. For aerosol and other spryable applications a variety of dispensing mechanisms are suitable and are known to those skilled in the art. For spray application, a gel-based formulation may be reduced in viscosity or an alternate formulation produced that is amenable to a spray application.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

Lotions and liniments according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

The compositions may be impregnated into transdermal patches, plasters, and wound dressings such as bandages or hydrocolloid dressings, preferably in liquid or semi-liquid form. By way of example only, topically applied compositions in accordance with the present invention may be formulated into, or with, face masks and scrubs, conditioning products such as lotions and creams, oils, shaving products such as creams and gels, skin washes, foams, bath and shower preparations such as oils and gels, moisturising products such as lotions, creams, gels and foams, anti-wrinkle products and anti-ageing products.

In particular circumstances, for example in the post surgical promotion of wound healing or the treatment of particular skin disorders, administration of compositions by injection, typically subcutaneous injection may be appropriate. Pharmaceutical forms suitable for injectable use include sterile aqueous solutions (where water soluble) or dispersions and sterile powders for the extemporaneous preparation of sterile injectable solutions. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compound(s) in the required amount in the appropriate solvent with various of the other ingredients enumerated above, as required, followed by filtered sterilisation. Generally, dispersions are prepared by incorporating the active compound(s) into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active compound(s).

The present invention contemplates combination therapies, wherein compositions as disclosed herein are coadministered with other suitable agents or treatments which may facilitate the desired therapeutic or cosmetic effect. For example, one may seek to aid wound healing with antibiotics, antimicrobial agents, or other wound healing agents in combination with compositions disclosed herein. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of agents. The agents may be administered in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Glyc-101™ Administration Following Laser Ablation of Skin

A study was carried out to compare the effects of gels containing 0.1% Glyc-101™ and 1.0% Glyc-101™, placebo and petroleum jelly (Vaseline®) when applied to laser-burned skin sites using female Goettingen mini-pigs.

The Glyc-101™ formulations tested comprised either 0.1% or 1% w/w Glyc-101™, in micro-particulate form, in a gel base consisting of ethanol, triethanolamine, Carbopol® 980 NF, titanium dioxide and purified water. The resulting composition is a highly viscous, aqueous gel suitable for topical administration. The Glyc-101™ (Glucoprime™) is poly-(1,3)-beta-D-glucopyranosyl-(1,6)-beta-D-glucopyranose, isolated from Saccharomyces cerevisiae using the procedure described and claimed in U.S. Pat. No. 6,242,594 (Kelly; the disclosure of which is incorporated herein by reference in its entirety).

One mini-pig was used to develop the procedures for determining the depth of laser ablation required to produce a mid-dermis burn that would approximate human wrinkle reduction in man. The skin on the mid-dorsal back was prepared by surgically scrubbing marked sites, each approximately 2×2 cm square. The laser setting was adjusted appropriately based on the experience of the operator. Laser ablation was conducted by making two passes over the first skin site. The same setting was used for the second and third sites but three and four passes were made, respectively. Punch biopsy samples were then taken from each site with each biopsy including some adjacent normal skin. The biopsy samples were prepared and preserved for histological evaluation. From evaluation of the biopsy samples, it was determined that two passes of the laser were required to affect a mid-dermis burn which approximates human wrinkle reduction in man.

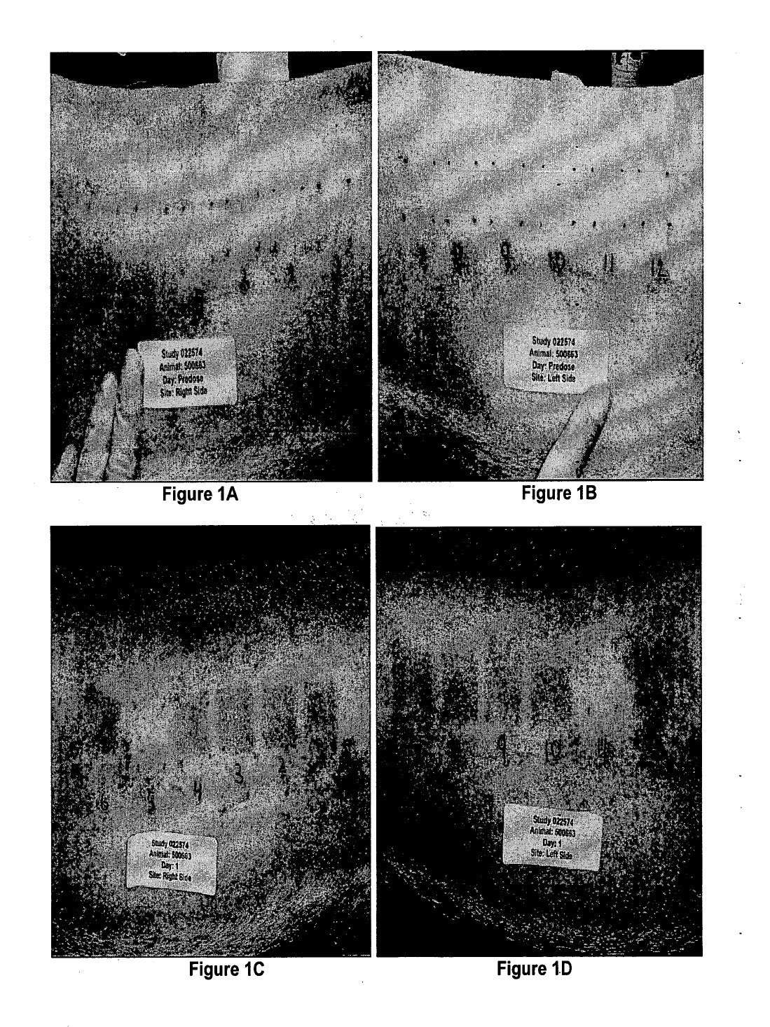

Four mini-pigs were used in the study on the effect of administration of glucan compositions (Glyc-101™) following laser ablation. The mini-pigs were prepared by marking and labelling twelve 2×2 cm test sites, 6 on each side of the spine (FIGS. 1A and 1B). The backs of the mini-pigs were clipped free of hair with electric clippers on the day prior to dermal laser ablation of the sections of skin. The application sites were on both sides of the spine about ⅓ of the way from the shoulder to the hip and approximately 4 cm lateral to the spine. The area was marked by tattoos outside of the area from which the skin was ablated. On Day 1, the first day of treatment, each animal was anesthetized and the skin area was surgically scrubbed and dermal ablation was conducted under aseptic procedures. Digital procedures were taken after ablation (FIGS. 1C and 1D). Animals received an injectable anesthetic, Telazol (Tiletamine-Zolazepam) and Xylazine IM prior to the preparation of the application site. After the ablation the char was removed with saline-saturated sterilized cotton gauze.

Following ablation two sites on each side of the mini-pig were treated by topical application with either 0.1% Glyc-101™ or 1.0% Glyc-101™; a placebo similar to that of the Glyc-101™ treated sites; or Vaseline®. A fifth site was left untreated and a sixth site was neither lasered nor treated and was used as a control. The dosing scheme for the sites 1 to 12 as shown in FIG. 1 is as follows: sites 1 and 7—1.0% Glyc-101™; sites 2 and 8—untreated laser; sites 3 and 9—placebo; sites 4 and 10—Vaseline; sites 5 and 11—normal skin; and sites 6 and 12—0.1% Glyc-101™. The formulations were uniformly applied over the application sites to a maximum thickness of 1 mm using a sterile-gloved finger and/or a disposable wooden tongue blade. A treatment chart was developed and followed to assure proper application of test materials.

Two of the four mini-pigs were treated daily for five consecutive days. The remaining two mini-pigs were treated on only the first, third and fifth days. Digital photographs of all sites were taken daily throughout the study and punch biopsy samples were removed under general anaesthesia at designated times (every day for the mini-pigs treated daily for five consecutive days and on the first, third and fifth for mini-pigs treated on the first, third and fifth days only) preserved in 10% formalin and held for histopathology analysis. A 6-mm size punch was used to remove tissue samples from each of the laser-ablated sites. The collected samples were taken at the edge of normal skin and the laser-ablated area so that normal and ablated tissue was obtained in each biopsy sample. On days 5 to 15, the progress of wound healing was observed and recorded daily.

Burn wound management entailed gentle wrapping of both sides of the mini-pig with sterile Tegaderm® foam placed directly over the test sites and held in place with Tensoplast® elastic tape. Wrapping prevented the animals from spreading the test compositions from one site to another. This dressing was applied daily for 5 days after which the sites were left open.

Each site was observed in an undisturbed state following laser ablation and/or each treatment. The conditions of the site were observed and recorded using definitions based on the Draize system (without scoring). Colour, swelling of the edges and surrounding skin, and changes in the site surface (scabbing, exudate, serious or purulent and scaling/flaking) were noted.

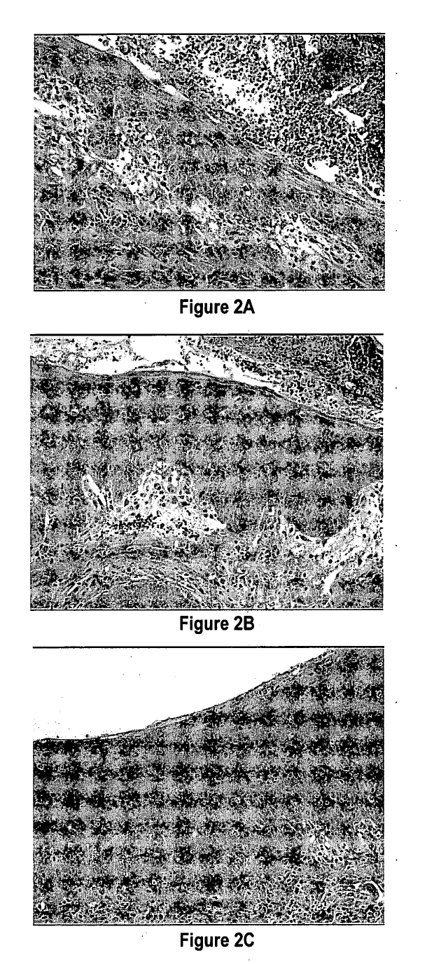

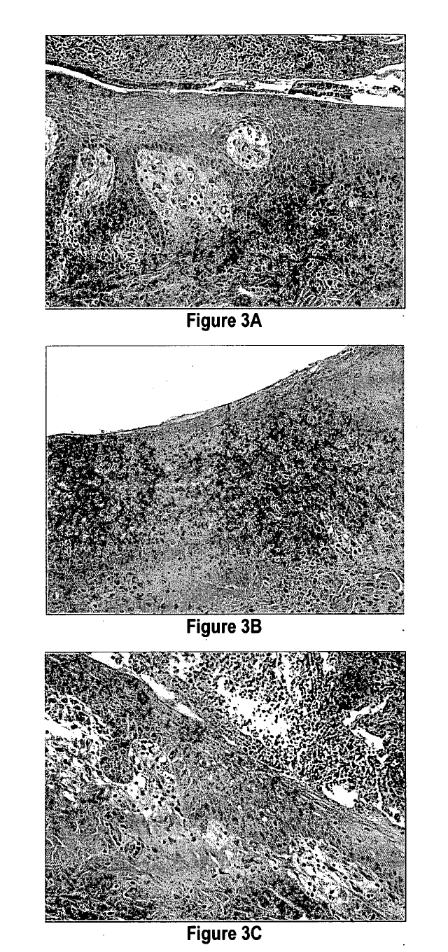

At Day 1 following laser ablation and treatment, nearly all sites were light pink with red areas within the 2×2-cm area. This appeared to be an inflammatory response to the laser burn and was noted in all sites receiving laser ablation. Over the course of the study, the observable findings were indistinguishable in treated test sites from normal healing signs. However histological examination of the punch biopsy samples showed significant cellular changes, depending on the test composition applied, the dose applied, and the frequency of application. Initially, laser ablation of the skin of the four mini-pigs resulted in coagulative necrosis of the epidermis and the superficial upper dermis. By Day 2, there was a fibrinopurulent inflammatory response to the tissue injury. Sites 4 (Vaseline® FIG. 2B) and 6 (0.1% Glyc-101™ FIG. 2C) showed changes in the appearance of the stratum corneum after 5 days of a daily application when compared to the untreated laser ablated control (FIG. 2A). The most marked increase in epidermal thickness occurred at the 0.1% Glyc-101™ test sites following daily administration for five days (FIG. 2C), most notably with regeneration in the stratum basale and stratum spinosum. Additionally, the sites treated on 5 consecutive days with 0.1% Glyc-101™ showed cellular regeneration sooner and in increased numbers of dermal growth cells than the sites treated on the first, third and fifth days only with 0.1% Glyc-101™. FIG. 3 shows the differences between epidermal regeneration in tissue from the test site treated with 0.1% Glyc-101™ daily for five days (FIG. 3B), the test site treated with 0.1% Glyc-101™ on the first, third and fifth days (FIG. 3A) and the untreated laser ablated control (FIG. 3C).

### Example 2

### Glyc-101™ Administration Following Split Skin Graft

A study was carried out to evaluate tissue response following split-skin graft preparation in the presence and absence of Glyc-101′. The effects of gels containing 0.1% Glyc-101™, 1.0% Glyc-101™ and placebo when applied to split-skin graft sites of female Goettingen mini-pigs were compared. The Glyc-101™ formulations tested comprised either 0.1% or 1% w/w Glyc-101™ as described in Example 1.

On Day 1, one mini-pig was used to develop the procedures necessary to achieve split-skin removal to the approximate depth of 0.012 inches (deep dermal with punctuate bleeding) on the mid-dorsal back. The skin on the mid-dorsal back was prepared by surgically scrubbing marked sites, each approximately 2.5×4 cm square. The selected mini-pig was anesthetized with appropriate anaesthetic agents and the skin area was aseptically cleaned. Split-skin removal of the treatment sites was performed using a Robbins electric, hand-operated dermatome. The same procedure was then used on two further mini-pigs to give a total of three mini-pigs used in the study.

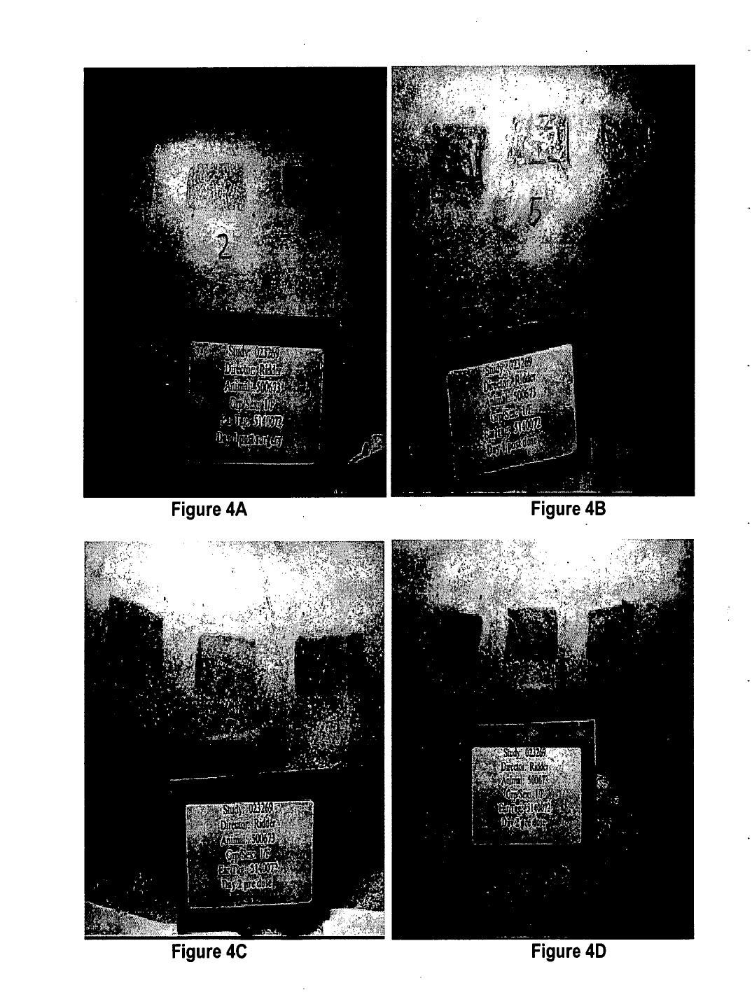

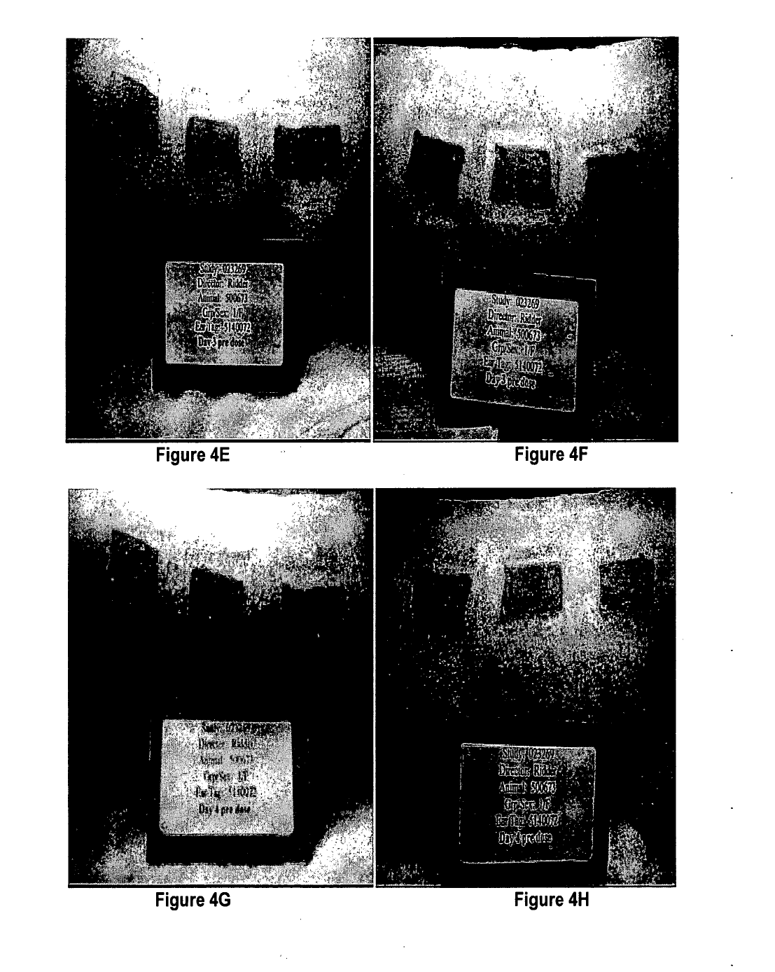

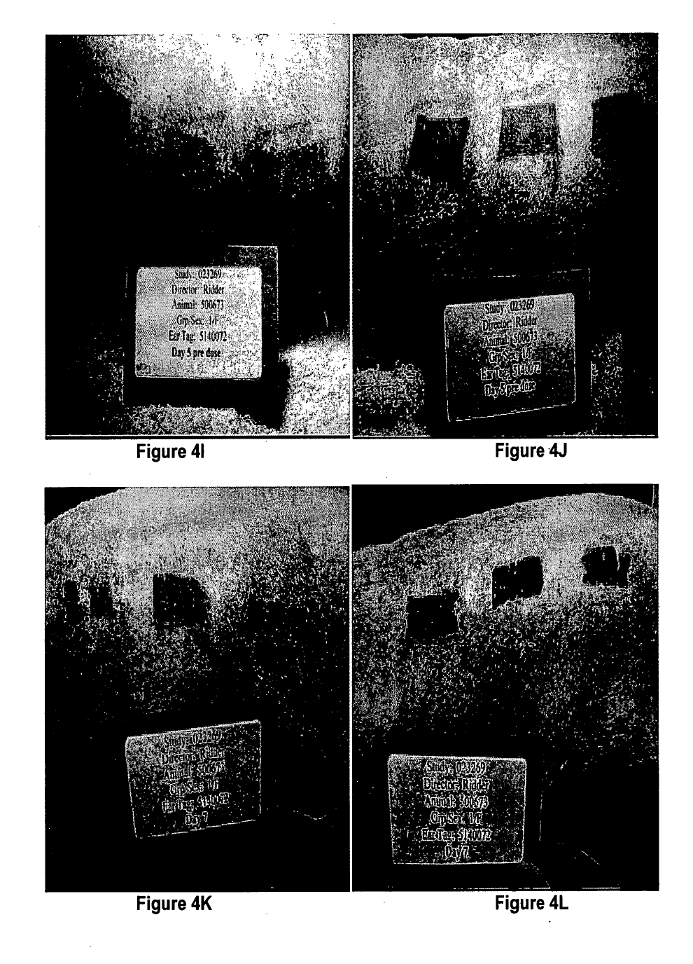

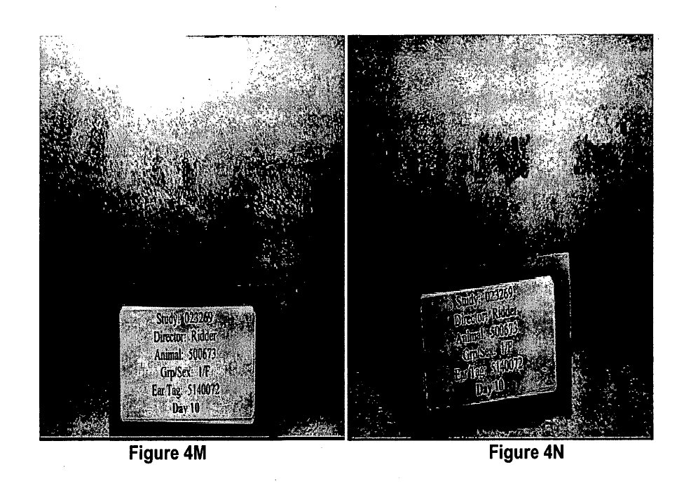

Following split-skin removal of the treatment sites two sites on each side of the mini-pig were treated by topical administration with either 0.1% Glyc-101™ or 1.0% Glyc-101™, or a placebo similar to that of the Glyc-101™ treated sites. The dosing scheme for the sites 1 to 6 as shown in FIG. 4 is as follows: sites 1 and 4—placebo; sites 2 and 5—0.1% Glyc-101™; and sites 3 and 6—1.0% Glyc-101™. The formulations were uniformly applied over the application sites to a maximum thickness of 1 mm daily for 5 days using a sterile-gloved finger and/or a disposable wooden tongue blade. A treatment chart was developed and followed to assure proper application of test materials.

Wound management following treatment of each site entailed gentle wrapping of both sides of the pig with sterile Tegaderm® foam placed directly over the sites and held in place with Tensoplast® elastic tape. This dressing was applied daily for 5 days, and then the sites were left open for an additional 5 days.

Digital photographs of both sides were taken on Days 1, 2, 3, 4, 5, 7, and 10 (FIG. 4). On Day 1, photographs were taken after split-skin removal (FIG. 4A) and after dosing (FIG. 4B). On Days 2 to 5, photographs were taken before dosing (FIGS. 4C to J). On Days 7 and 10 (FIGS. 4K to N), photographs were taken in the morning (no dosing after Day 5). Punch biopsies were taken daily on Days 1 to 5 and on Day 10 from sites 4, 5 and 6. On Day 1 the punch biopsies were taken after split-skin removal (all punch biopsies were obtained while the pig was under general anaesthesia). A 6-mm punch was used to remove tissue samples from each site.

Following split-skin graft removal and/or treatment, the test sites were observed and recorded (without scoring). Colour, swelling and changes in the site surface (scabbing, exudate (serious or purulent), and scaling/flaking) were noted.

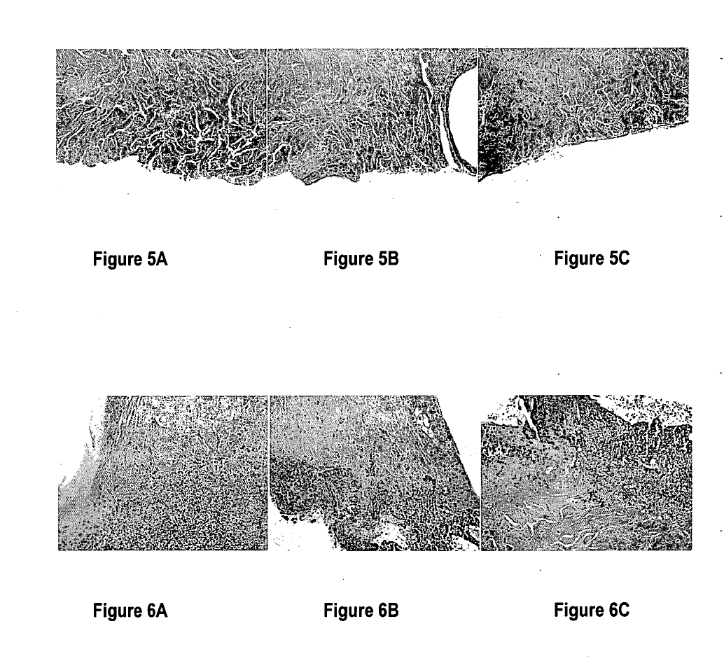

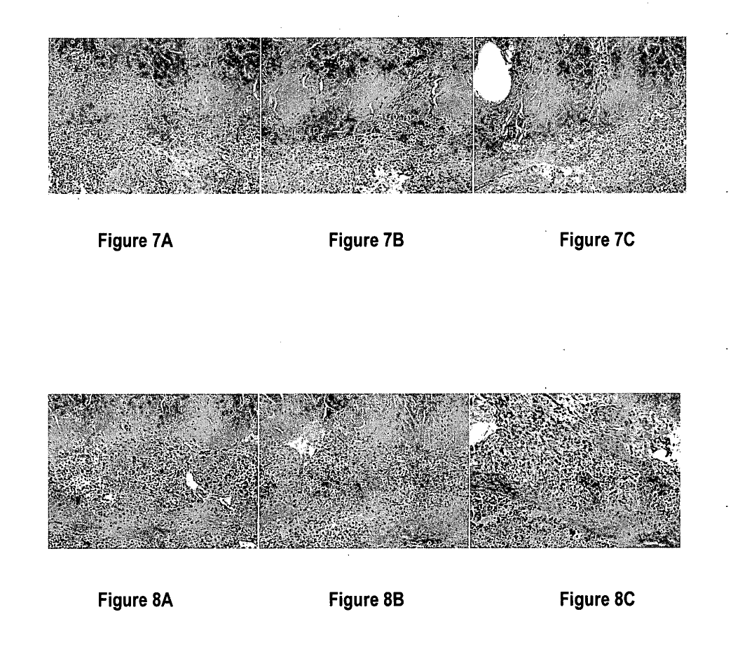

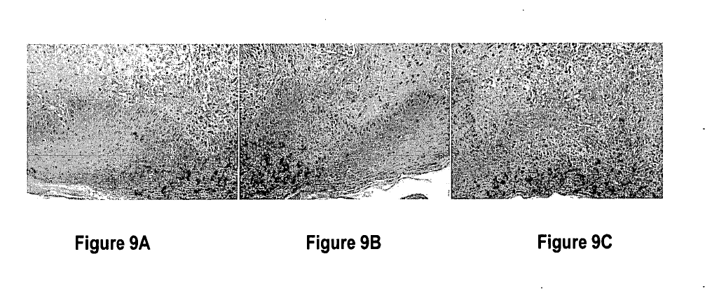

At Day 1 histological studies showed that the split-skin graft removal of the skin of three female Gottingen mini-pigs resulted in nearly total removal of the epidermis except for epidermis associated with the hair follicles and evidence of coagulative necrosis of the epidermis and the superficial upper dermis (FIGS. 5A to 5C). At Days 2 and 3 (FIGS. 6A to 6C and FIGS. 7A to 7C), there was an inflammatory response (inflammatory phase) to the tissue injury with focal aggregations of degenerative polymorphonuclear neutrophils with necrotic debris and a significant amount of edema between the basement membrane and the underlying dermis. Regenerative processes (proliferating phase) starting in the stratum basale were evident minimally on Day 3 (FIGS. 7A to 7C) in all mini-pigs and with all treatment sites. By Day 5 (FIGS. 9A to 9C), there was marked progression of regeneration of the epidermis, most notably the stratum basale, stratum spinosum and stratum corneum in treatment sites of all three mini-pigs with appearance of moderate stratum lucidum with keratohyalin granules and mild stratum corneum in treatment site 6 (FIG. 9C) to which 1.0% GLYC-101 was applied, in all three mini-pigs. Complete reepithelialization of the epidermis was observed at Day 10 (FIGS. 10A to 10 C).

In summary, based on the daily gross findings all treated sites were indistinguishable from normal healing signs. Histologic examination of the punch biopies showed similar changes through Day 3; however, a clearly greater regenerative response was evident with 1% GLYC-101 compared to 0.1% GLYC-101 or placebo based on changes in the stratum lucidum and stratum corneum at Day 5 of treatment.

### Example 3

### Exemplary Compositions for Treatment

By way example only suitable glucan formulations for use in accordance with embodiments of the invention are outlined below. The following are to be construed as merely illustrative examples of compositions and not as a limitation of the scope of the present invention in any way.

### Gel Composition

A composition for topical administration may be an aqueous gel containing glucan in microparticulate form, in a composition comprising ethanol, triethanolamine, Carbopol® 980 NF, titanium dioxide and purified water. The glucan may be present in a 0.1% or 1% w/w ratio to produce the product. The resulting composition is a highly viscous, aqueous gel suitable for topical administration.

### Ointment Composition

A typical composition for delivery as an ointment includes the desired amount of glucan, together with white soft paraffin to 100.0 g, dispersed to produce a smooth, homogeneous product.

### Topical Cream Composition

A typical composition for delivery as a topical cream is outlined below:

| Glucan | (as desired) | |
|---|---|---|
| Polawax GP 200 | 25.0 | g |
| Lanolin Anhydrous | 3.0 | g |
| White Beeswax | 4.5 | g |
| Methyl hydroxybenzoate | 0.1 | g |
| Deionised & sterilised Water | to 100.0 | g |

The polawax, beeswax and lanolin are heated together at 60° C., a solution of methyl hydroxybenzoate is added and homogenisation achieved using high speed stirring. The temperature is then allowed to fall to 50° C. The glucan is then added and dispersed throughout, and the composition is allowed to cool with slow speed stirring.

### Topical Lotion Composition

A typical composition for delivery as a topical lotion is outlined below:

| Glucan | (as desired) | |
|---|---|---|
| Sorbitan Monolaurate | 0.8 | g |
| Polysorbate 20 | 0.7 | g |
| Cetostearyl Alcohol | 1.5 | g |
| Glycerin | 7.0 | g |
| Methyl Hydroxybenzoate | 0.4 | g |
| Sterilised Water | about to 100.00 | ml |

The methyl hydroxybenzoate and glycerin are dissolved in 70 ml of water at 75° C. The sorbitan monolaurate, polysorbate 20 and cetostearyl alcohol are melted together at 75° C. and added to the aqueous solution. The resulting emulsion is homogenised, allowed to cool with continuous stirring and the glucan is added as a suspension in the remaining water. The whole suspension is stirred until homogenised.

1. A method for the treatment of a skin wound or lesion, or connective tissue damage or injury, the method comprising administering to a subject an effective amount of a glucan composition comprising from about 0.01% (10 μg/ml) to about 10% (10 mg/ml) glucan.

2. The method of claim 1, wherein the glucan composition comprises from about 0.02% (20 μg/ml) to about 5% (5 mg/ml) glucan, from about 0.05% (50 μg/ml) to about 2% (2 mg/ml) glucan, or from about 0.05% (50 μg/ml) to about 1% (1 mg/ml) glucan.

3-4. (canceled)

5. The method of claim 1, wherein the glucan composition comprises about 0.1% (100 μg/ml) glucan, or about 1% (1 mg/ml) glucan.

6-8. (canceled)

9. The method of claim 1, wherein the glucan is a microparticulate branched beta-(1,3)(1,6) glucan.

10. The method of claim 1, wherein the glucan is microparticulate poly-(1,3)-beta-D-glucopyranosyl-(1,6)-beta-D-glucopyranose.

11. The method of claim 1, wherein the glucan is administered topically to the skin of the subject.

12. (canceled)

13. The method of claim 1, wherein the glucan is administered in a dressing or bandage into which the glucan has been incorporated.

14. The method of claim 1, wherein the administration is at least once daily, or at least once daily for at least five days.

15. (canceled)

16. The method of claim 1, wherein the skin wound or lesion is a result of laser or chemical peeling.

17. The method of claim 16, wherein the glucan composition comprises about 0.1% (100 μg/ml) glucan.

18. The method of claim 1, wherein the subject requires reconstructive or cosmetic surgery and wherein the glucan composition is administered before, during or after said surgery.

19. The method of claim 1, wherein the skin wound or lesion is a result of reconstructive or cosmetic surgery.

20. The method of claim 19, wherein the reconstructive or cosmetic surgery comprises a skin graft procedure.

21. (canceled)

22. The method of claim 20, wherein the glucan composition comprises about 1% (1 mg/ml) glucan.

23-28. (canceled)

29. A method for promoting tissue regeneration and/or promoting wound healing in a subject, the method comprising administering to a subject an effective amount of a glucan composition comprising from about 0.01% (10 μg/ml) to about 10% (10 mg/ml) glucan.

30. The method of claim 29, wherein the tissue is skin.

31. (canceled)

32. A method for promoting the growth of dermal tissue in a tissue culture comprising the same, comprising exposing the tissue culture to an effective amount of a glucan composition comprising from about 0.01% (10 μg/ml) to about 10% (10 mg/ml) glucan.

33. The method of claim 32, wherein the dermal tissue is grown to produce tissue for grafting onto a burn, surgical wound, acute wound or chronic wound.

34. The method of claim 32, wherein the dermal tissue is derived from a subject in need of a tissue graft.

35-36. (canceled)

37. A unit dose treatment product for the treatment of a skin wound or lesion, or connective tissue damage or injury, or the promotion of healing thereof, the product comprising multiple individual containers each containing a unit dose of a glucan composition, the glucan composition comprising from about 0.01% (10 μg/ml) to about 10% (10 mg/ml) glucan, and wherein the product is designed for the sequential application of the components of the individual containers, preferably once a day for at least 5 consecutive days.
